(19) United States
(12) Patent Application Publication
(10) Pub. No.: US 2016/0145300 A1
(43) Pub. Date: May 26, 2016

(54) ARTIFICIAL DECAPEPTIDE, MEDICATION AND METHOD OF INDUCING BREEDING OF CRUSTACEANS

(71) Applicant: National Pingtung University of Science and technology, Pingtung (TW)

(72) Inventors: Ying-Nan Chen, Pingtung County (TW); Song-Cen Wong, Pingtung County (TW); Meng-Ling Lu, Pingtung County (TW); Shu-En Chi, Pingtung County (TW)

(21) Appl. No.: 14/658,247

(22) Filed: Mar. 16, 2015

(30) Foreign Application Priority Data
Nov. 25, 2014 (TW) 103140829

(51) Int. Cl. C07K 7/06; A01K 61/00

(52) U.S. Cl. CPC C07K 7/06; A01K 61/005

(57) ABSTRACT

The present invention provides an artificial decapeptide for inducing the development of gonad in crustacean, a medication including the artificial decapeptide and a method of inducing the gonad development of crustacean. The method includes administrating an effective amount of an artificial decapeptide to a crustacean, thereby promoting gonad development of crustaceans, wherein the artificial decapeptide includes a sequence set forth in SEQ ID NO: 2.

### BACKGROUND OF THE INVENTION

1. Field of the Invention

The present invention relates to an artificial decapeptide, a medication and a method of inducing breeding of crustaceans, and more particularly to an artificial decapeptide and a medication on a basis of a gonadotropin releasing hormone of vertebrate, and a method of inducing breeding of crustaceans by using the artificial decapeptide.

2. Description of the Prior Art

Currently studies on reproductive endocrine of invertebrates (such as crustaceans) are quite limited, and people skilled in the art generally believe the reproductive endocrine system and mechanism thereof are far different from that of vertebrates. For example, it is only clear that vitellogenesis inhibiting hormone (VIH) in female crustaceans, a kind of neuropeptides secreted from eyestalk, will inhibit the synthesis and the accumulation of a precursor of vitellogenin in ovum (through endocytosis), and vitellogenesis stimulating hormone (VSH) in female crustaceans, another kind of neuropeptides secreted from thoracic ganglion, will promote the synthesis and the accumulation of such precursor of vitellogenin, in ovum. However, precise structure and material characterizes of such vitellogenesis stimulating hormone are still vague in the art. On the other hand, less information is known about regulatory mechanism of male crustaceans. For example, it is only clear that androgenic gland hormone, which is secreted from androgenic gland, may play important role in sexual differentiation and gonad development. However, the entire mechanisms and endocrine systems related to spermatophore maturation in crustaceans are still unknown.

Although current techniques may utilize methods such as biogenic amine (BA), serotonin (5-HT) or eyestalk ablation to induce the gonad development of aquafarmed crustaceans, it is still poor in controlling the gonad maturation of such aquafarmed crustaceans. Furthermore, the aforementioned techniques, for example eyestalk ablation, may further lead to imbalanced physiological responses, such as shelling in mated females or high mortality. Thus, most of high economic crustacean organisms may not be properly bred under current aquafarming environments, and therefore, it is quite limited in obtaining gonadal mature breeds or mass producing gonadal mature crustaceans.

### SUMMARY OF THE INVENTION

It is one of the primary objectives of the present invention to provide an artificial decapeptide and a medication of inducing breeding of crustaceans, which can effectively and extendedly promote gonad development of mature crustaceans (including male and female), thereby inducing spawning.

It is one of the primary objectives of the present invention to provide a method of inducing breeding of crustaceans, which can facilitate breeding and mass-producing the breeds of crustaceans, so as to produce gonad-mature crustaceans which can be used for industrial purposes.

To achieve the purposes described above, one embodiment of the present invention provides an artificial decapeptide of inducing breeding of crustaceans, which includes a sequence set forth in SEQ ID NO: 2.

To achieve the purpose described above, one embodiment of the present invention provides a medication of inducing breeding of crustaceans, including the aforementioned artificial decapeptide and at least one excipient.

To achieve the purpose described above, one embodiment of the present invention provides a method of inducing breeding of crustaceans, including administrating an effective amount of an artificial decapeptide to a crustacean, thereby promoting gonad development of the crustacean.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic line chart illustrating the maturity of spermatophore in male shrimps (Litopenaeus vannamei).

FIG. 2 is a schematic bar chart illustrating the maturity of spermatophore in male shrimps (Litopenaeus vannamei).

### DETAILED DESCRIPTION

In the following description, numerous specific details, including contents and the purposed efficacy of the present invention, are given to provide a thorough understanding of the invention. It will, however, be apparent to one skilled in the art that the invention may be practiced without these specific details. The figures, which comprise a part of the present invention, are drawn to provide ease of explanation and for illustrating a preferable embodiment of the present invention. The embodiment below has been disclosed and explained so that a person who has ordinary skill in the art can understand and utilize the following teachings after reading the following. Modifications of the method of the present invention are also possible, and the method detailed therein may also be performed to form an alternative structure based on the present embodiment. Therefore, the following detailed description is not to be taken in a limiting sense, with the embodiment therein being defined by what is claimed instead.

One embodiment of the present invention provides a method of inducing breeding of crustaceans, including the following steps. First of all, an artificial decapeptide is provided, and which includes a sequence set forth in SEQ ID NO: 2. Next, an effective amount of the artificial decapeptide is administrated to a crustacean, thereby promoting the gonad development of the crustacean. Precisely, the artificial decapeptide may facilitate the maturation of spermatophore, as well as increase the quantity of sperm in male crustaceans. On the other hands, the artificial decapeptide may also promote ovarian development and increase spawning in female crustaceans.

The method of the present invention is primarily applied in crustaceans, and more particular high economic organisms, such as organisms in Brachyuran suborder, including mitten crab (Eriocheir rectus), land crab, crimson crab (Ranina ranina), and Chinese mitten crab (Eriocheir sinensis); organisms in Macrura suborder, such as prawn of Penaeus; and organisms in Anomura suborder, such as hermit crab (Paguridae) and coconut crab (Birgus latro). It is noted that, such organisms in decapoda order share similar body tissues and physiologic mechanisms, and the only difference therebetween is mainly in external appearance. Precisely, such organisms in decapoda order all have the same or similar zoea stage, and only a part of species have to go through a metamorphosis state during growing, thereby leading to segment atrophy which may result in the aforementioned difference. However, such organisms in decapoda order still share highly relative gonad secretion systems.

In one embodiment of the present invention, the artificial decapeptide is on the basis of naturally occurring gonadotropin releasing hormone II (GnRH-II) of vertebrates, such as chicken, duck, and goose, and the artificial decapeptide may have a sequence as set forth in SEQ ID NO: 1. Precisely, the artificial decapeptide may include 10 amino acids which is designed based on naturally occurring chicken GnRH-II. The method of forming the artificial decapeptide, for example, includes artificially synthesizing the decapeptide via a solid-phase peptide synthesis method. In comparison with the naturally occurring chicken GnRH-II, the glutamic acid (Glu) of the artificial decapeptide in N-terminal domain does not cyclized to form pyro-Glu, and the glycine (Gly) in C-terminal domain does not lactamize to form Gly-NH₂. In other words, the amino acids of the artificial decapeptide are native and unblocked.

After these, an effective amount of the artificially decapeptide is administrated to a crustacean, wherein, the effective amount of the artificially decapeptide may be 10 nanograms (ng) to 1000 ng, preferably 50 ng, per gram of body weight for each administrating. In one embodiment of the present embodiment a sustainable releasing dosage of the artificial decapeptide may be optionally prepared and administrated to the crustacean, so as to simplify the administration process. A method of preparing the sustainable releasing dosage of the artificially decapeptide, for example, may include formulating a great amount of the artificially decapeptide in an excipient, such as an excipient including oil. For example, 100 ng to 2000 ng of the artificially decapeptide may be formulated in 50 microliter (μl) to 200 μl of the excipient. With such arrangement, while the sustainable releasing dosage of the artificially decapeptide is administrated to the crustacean, the artificially decapeptide therein may be allowed to progressively release in the crustacean, thereby extending the inducing effect to the crustacean. Thus, in another embodiment of the present invention, the sustainable releasing dosage of the artificially decapeptide may be administrated to the crustacean every three to six months, preferably every four months, but not limited thereto. In other embodiment of the present invention, the artificially decapeptide may also be firstly formulated in saline, to forma solvent, and then, mixed with the excipient, wherein a volume ratio between the solvent and the excipient may be 1:1 to 1:3. Furthermore, in another embodiment, the excipient may include mineral oil, such as glycerol or incomplete adjuvant; vegetable oil, such as peanut oil, olive oil or coconut oil; fat adhesive, such as methylcellulose; or a composite of the aforementioned oil.

It is worth mentioning that, an entire volume of the solution (including the artificial decapeptide) which is allowable to be given to the crustacean is substantially between 50 μl and 200 μl, every three to four months, but is not limited thereto. Also, the solution is preferably injected into a body cavity of the crustacean, for example, a cavity of carapace which is located at a bottom part of a rostrum in a macryra organism, such as shrimp, or a cavity of side carapace in a brachyura organism, such as crab. With such arrangement, a great amount of the artificial decapeptide, as well as a great volume of the solution, may be allowable to be given to the crustacean in single injection, and which will not lead to the stress and pressure to such crustacean.

Through the aforementioned embodiments, it is noted that the method of inducing breeding of crustaceans in the present invention provides the artificial decapeptide which is artificially synthesized on the basis of naturally occurring chicken GnRH-II and includes a sequence set forth in SEQ ID NO: 2, and further administrates such artificial decapeptide to a crustacean, thereby promoting the gonad development of the crustacean. Furthermore, it is worth mentioning that, the method of the present invention is actually across the barrier between vertebrates and invertebrates, since an analogue of gonadotropin releasing hormone derived from vertebrates is successfully applied to invertebrates (crustaceans) for promoting gonad development thereof, such as facilitating the maturation of spermatophore and increasing the quantity of sperm in male crustaceans, and promoting ovarian development and increasing spawning in female crustaceans.

TABLE 1

| | groups | |
|---|---|---|
| tests | artificial decapeptide | control |
| spermatophore | 0.0217 ± 0.007 | 0.0181 ± 0.007 |
| weight (g) | | |
| sperm count (10⁵/per | 68.5 ± 18.14 | 24.89 ± 12.64 |
| spermatophore) | | |

TABLE 2

| | |
|---|---|
| ovarian | |
| development | groups |

| stage | artificial decapeptide | control |
|---|---|---|
| 0 | 0%^{b} | 20%^{b} |
| 0.5 | 20%^{b} | 40%^{b} |
| 1 | 0%^{b} | 0%^{b} |
| 1.5 | 0%^{b} | 0%^{b} |
| 2 | 0%^{b} | 0%^{b} |
| 2.5 | 0%^{b} | 0%^{b} |
| 3 | 0%^{b} | 0%^{b} |
| spawn | 80%^{b} | 40%^{b} |

^{a}ovarian development stage is identified based on external characteristics and maturity of ovarian, and which may be primary divided into four stages. While the maturity of ovarian between two stages may be further defined as stage 0.5, stage 1.5 and stage 2.5. Generally, in an aquafarming environment, if a female shrimp has ovarian development beyond stage 3, such female shrimp may spawn soon.
^{b}percentage relative to overall Litopenaeus vannamei in the test

TABLE 3

groups

| | artificial | |
|---|---|---|
| spawning times^{c} | decapeptide | control |
| 0 | 20%^{b} | 60%^{b} |
| 1 | 20%^{b} | 40%^{b} |
| 2 | 20%^{b} | 0%^{b} |
| 3 | 0%^{b} | 0%^{b} |
| 4 | 40%^{b} | 0%^{b} |

^{b}percentage relative to overall Litopenaeus vannamei in the test
^{c}The test of spawning times is carried out periodically in 8-16 weeks after administrating the solution (artificial decapeptide) to Litopenaeus vannamei. However, data obtained in first 7 weeks (namely 7 weeks after administrating the solution) have been precluded, due to avoiding external disturbances.

In the following, functions and advantages of the artificial decapeptide, such as promoting gonad development of the crustaceans, will be further illustrated and demonstrated. In the present test, the artificial decapeptide of the present invention is exemplified on numbers of Litopenaeus vannamei (Decapoda: Macrura). However, people in the art should easily realize the present invention is not limited thereto.

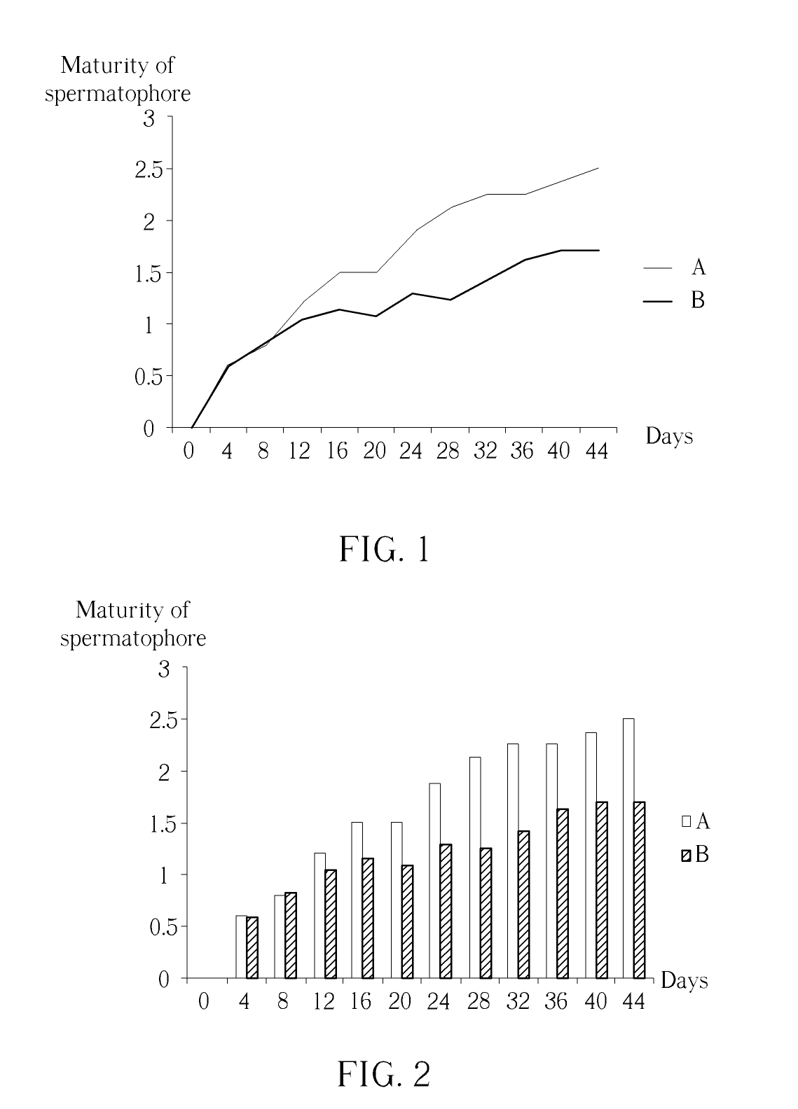

In the present test, the artificial decapetide is firstly formulated in the aforementioned sustainable releasing dosage and then administrated to each Litopenaeus vannamei. After that, the spermatophore development of each male shrimp is determined and recorded every 4 days after the administration, through observing the external characteristics spermatophore. Also, the spermatophore weight, as well as the sperm count, is precisely measured 44 days after the administration. On the other hand, the ovarian development of each female shrimp is also determined and recorded weekly 2-4 months after the administration through observing the external characteristics and the maturity of ovarian. Furthermore, the spawning times of each female shrimp may be also recorded. Please refer to TABLEs 1-3 and FIGS. 1-2, which illustrate gonadal promoting effects of the artificial decapeptide either on male or female shrimps (Litopenaeus vannamei). Wherein, TABLE 1 and FIGS. 1-2 show the maturity of spermatophore in male shrimps, including the development stage, spermatophore weight and sperm count per spermatophore, after the artificial decapeptide is administrated, in comparison with untreated male shrimps (control; marked as B in FIGS. 1-2), respectively. TABLE 2 shows the maturity of ovarian in female shrimps after the artificial decapeptide is administrated, in comparison with untreated female shrimps (control; marked as B in FIGS. 1-2); and TABLE 3 shows the spawning times in female shrimps after the artificial decapeptide is administrated, in comparison with untreated female shrimps (control; marked as B in FIGS. 1-2).

As shown in TABLE 1, FIGS. 1-2 in which the functions on male crustacean organisms are exemplified, the artificial decapeptide of the present invention can effectively facilitate the maturation of spermatophore. Precisely, in comparison with control shrimps (shown as B in FIGS. 1-2), the artificial decapeptide sufficiently improve the spermatophore weight and the sperm count of spermatophore in male shrimps, and also, the spermatophore development of such male shrimps may reach stage 2.5 in 44 days after the administration. In other words, in 44 days after the artificial decapeptide is administrated, it is clear to observe beige, sack-like structures of spermatophore. Also, as shown in TABLE 2, in which the functions on female crustacean organisms are exemplified, the artificial decapeptide of the present invention can also sufficiently promote the ovarian development. Precisely, in comparison with control shrimps(shown as B in FIGS. 1-2), substantially 80% of female shrimps have ovarian developed to the spawning stage after the artificial decapeptide is administrated, and only substantially 40% of untreated female shrimps have ovarian developed to the spawn stage. Furthermore, as shown in TABLE 3, while being developed to the spawning stage, substantially 40% of female shrimps may spawn four times, 20% of female shrimps may spawn twice, and another 20% many of female shrimps may spawn once after the artificial decapeptide is administrated. However, all of the untreated female shrimps may only spawn only once while those have developed to the spawning stage. Thus, the artificial decapeptide of the present invention, cannot only promote the ovarian development in female crustaceans, but also increase the number of spawning times.

Furthermore, while comparing to current techniques, such as unilateral eyestalk ablation or giving of biogenic amine or serotonin, the artificial decapeptide of the present invention may achieve better long-term effects. For example, the current techniques may only induce fewer times of spawning and the effective period only lasts for around one month, and the artificial decapeptide of the present invention may induce four spawnings and the effective period may be between two and four months. Thus, through utilizing the artificial decapeptide of the present invention, it is sufficient to dramatically increase the quality and the quantity of spawn in crustaceans.

In summary, the artificial decapeptide of the present invention is capable of being applied to any male or female crustaceans for inducing gonad development and sexual maturity, thereby successfully farming mature breeds, more particular high economic decapoda organisms (such as shrimps and crabs) . Thus, the method of the present invention is allowable to be used in aquafarming or other industries. Although the aforementioned embodiments are mainly exemplified on Litopenaeus vannamei (Decapoda: Macrura), people skilled in the art should easily realize the present invention may also be reasonable utilized on other crustaceans which is not redundantly described herein.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of inducing breeding of crustaceans, comprising:
administrating an effective amount of an artificial decapeptide to a crustacean, being configured to promote gonad development of the crustacean, wherein the artificial decapeptide comprises a sequence set forth in SEQ ID NO: 2.

2. The method of inducing breeding of crustaceans of claim 1, wherein, the effective amount of an artificial decapeptide ranges between 10 nanogram (ng) and 1000 nanogram per gram of body weight.

3. The method of inducing breeding of crustaceans of claim 1, further comprising:
preparing the artificial decapeptide by mixing with an excipient, and delivering the prepared artificial decapeptide to the crustacean, wherein the excipient comprises an oil.

4. The method of inducing breeding of crustaceans of claim 3, further comprising:
formulating the artificial decapeptide with saline before mixing with the excipient, to obtain a solvent; and
mixing the solvent and the excipient.

5. The method of inducing breeding of crustaceans of claim 4, wherein, a ratio between the solvent and the excipient is between 1:1 and 1:3.

6. The method of inducing breeding of crustaceans of claim 4, wherein, the oil is selected from a group consisting of vegetable oil, mineral oil, and fat adhesive.

7. An artificial decapeptide for inducing breeding of crustaceans, comprising a sequence set forth in SEQ ID NO: 2.

8. A medication for inducing breeding of crustaceans, comprising:
an artificial decapeptide, comprising a sequence set forth in SEQ ID NO: 2; and
at least one excipient.

9. The medication for inducing breeding of crustaceans of claim 8, wherein, an effective amount of the artificial decapeptide ranges between 10 nanograms and 1000 nanograms per gram of body weight.

10. The medication for inducing breeding of crustaceans of claim 8, wherein the excipient comprises an oil which is selected from a group consisting of vegetable oil, mineral oil, and fat adhesive.


SEQUENCE LISTING (2 sequences)

SEQ ID NO: 1 - protein, 10 aa, Gallus gallus
EHWSHGWYPG

SEQ ID NO: 2 - protein, 10 aa, Artificial Sequence
  unblock N-terminal and C-terminal
EHWSHGWYPG